(19) United States
(12) Patent Application Publication
(10) Pub. No.: US 2026/0258510 A1
(43) Pub. Date: Sep. 3, 2026

(54) METAGENOMIC NEXT-GENERATION SEQUENCING OF MICROBIAL CELL-FREE NUCLEIC ACIDS IN SUBJECTS WITH LYME DISEASE

(71) Applicants: Karius, Inc., Redwood City, CA (US); The Research Foundation for The State University of New York, Albany, NY (US)

(72) Inventors: Asim A. Ahmed, Redwood City, CA (US); Carine Ho, Redwood City, CA (US); Desiree D. Hollemon, Redwood City, CA (US); Andrew Handel, Albany, NY (US); Christy Beneri, Albany, NY (US)

(21) Appl. No.: 19/656,884

(22) Filed: Apr. 23, 2026

Related U.S. Application Data
(63) Continuation of application No. 18/365,876, filed on Aug. 4, 2023, now abandoned.
Application No. 18/365,876 is a continuation of application No. PCT/US2022/016232, filed on Feb. 11, 2022.
(60) Provisional application No. 63/148,858, filed on Feb. 12, 2021.

(51) Int. Cl. C12Q 1/689; C12N 15/10

(52) U.S. Cl. CPC C12Q 1/689; C12N 15/1034; C12Q 2600/112

(57) ABSTRACT

What is disclosed herein is a method of detecting and treating Lyme disease, particularly Lyme arthritis. The method includes metagenomic next-generation sequencing (mNGS) of plasma microbial cell-free DNA (mcfDNA) to detect and quantify Borrelia spp. in the circulation. Also disclosed is a method of treating Lyme arthritis by treating the patient with an anti-microbial treatment until Borrelia mcfDNA is lowered to below detectable levels, such as less than 1 molecule per microliter of plasma.

### CROSS-REFERENCE

This application is a continuation application of U.S. patent application Ser. No. 18/365,876, filed Aug. 4, 2023, which is a continuation application of International Patent Application No. PCT/US2022/016232, filed on Feb. 11, 2022, which claims the benefit of U.S. Provisional Patent Application 63/148,858 filed on Feb. 12, 2021, each of which application is incorporated herein by reference in its entirety.

### BACKGROUND

Lyme disease, also known as Lyme borreliosis, is a vector-borne disease caused by the Borrelia bacterium and is generally spread by ticks. Lyme disease is the most common zoonotic infection in the United States. For decades, the diagnostic standard of care has remained a combination of clinical signs and symptoms with serology (per Centers for Disease Control and Prevention (CDC) guidance), despite shortcomings of antibody-based testing. As with any serologic test, sensitivity is low early in infection. This renders the test generally unhelpful at the time of an erythema migrans (EM), the infection's earliest and most frequent, though often missed and potentially non-specific, manifestation. The clinical diagnosis of acute localized Lyme can be quite challenging given the reliance on the skills of the clinician and issues of subjectivity in the recognition of the EM rash. In addition, there may be other etiological agents which produce phenotypically similar rashes. Once formed, anti-Borrelia burgdorferi antibodies may persist for years, with evidence of seropositivity 10-20 years following infection. As a result, serology inconsistently distinguishes prior from new infections and is generally unhelpful as a test-of-cure.

The search for a reliable clinical tool to directly detect B. burgdorferi has been largely unsuccessful due to poor sensitivity. Studies of blood culture sensitivity, for example, found a wide, poorly reproducible sensitivity range from 27-94%. Polymerase chain reaction (PCR) sensitivity varies by anatomical site, exceeding 90% in the synovial fluid of patients with Lyme arthritis, but only 18% in serum from patients with a single EM.

### SUMMARY

Disclosed herein in an aspect is a method of detecting Borrelia spp. in a subject, the method comprising: collecting one or more samples (e.g., blood, plasma, serum samples) from the subject at a time when the subject does not have an erythema migrans (EM) rash and wherein the one or more blood samples comprise microbial cell-free nucleic acids (mcfNA); and detecting mcfNA from Borrelia spp. in the one or more blood samples. In some embodiments, mcfNA from Borrelia spp comprises microbial cell-free DNA from Borrelia spp. In some embodiments, one or more blood samples are one or more plasma samples. In some embodiments, the one or more samples comprise cell-free nucleic acids. In some embodiments, the method comprises attaching the cell-free nucleic acids (e.g., cfNA, cfDNA, cfRNA) to nucleic acid adapters to prepare a sequencing library comprising the mcfNA. In some embodiments, the method comprises attaching the mcfNA to nucleic acid adapters to prepare a sequencing library comprising the mcfNA. In some embodiments, the method comprises performing next-generation or metagenomic sequencing of nucleic acids (e.g., cell-free nucleic acids, cell-free DNA, cell-free RNA) from the one or more samples. In some embodiments, the method comprises generating sequence reads from the sequencing library comprising the mcfNA, aligning the sequence reads to Borrelia spp. genomic sequences in a reference data set to obtain aligned sequence reads, and identifying the Borrelia spp. based on the aligned sequence reads. In some embodiments, the method comprises administering a therapeutic treatment to the subject to treat a Borrelia spp. infection. In some embodiments, the therapeutic treatment comprises a Borrelia-directed therapy. In some embodiments, the Borrelia-directed therapy comprises at least one therapy selected from the group consisting of: doxycycline, amoxicillin, cefuroxime axetil, ceftriaxone, and cefotaxime. In some embodiments, the method comprises spiking the one or more blood samples with a known concentration of synthetic DNA. In some embodiments, the method comprises spiking the one or more plasma samples with a known concentration of synthetic DNA. In some embodiments, a concentration of the Borrelia mcfNA per microliter of blood is measured. In some embodiments, a concentration of Borrelia mcfNA per microliter of blood is greater than a threshold amount. In some embodiments, the subject has arthritis. In some embodiments, the subject has arthritis of a joint. In some embodiments, the joint comprises at least one joint selected from the group consisting of knee, elbow, temporomandibular joint, and hip. In some embodiments, the subject is blood culture negative for Borrelia at the time of the collecting of the one or more blood samples. In some embodiments, the subject is negative for Borrelia when measured by a polymerase chain reaction (PCR) test of a sample of blood from the subject. In some embodiment, the subject is negative for Borrelia when measured by a polymerase chain reaction (PCR) test of a sample of synovial fluid from the subject. In some embodiments, the subject was bitten by a tick carrying Borrelia bacteria at least 6 months prior to the collecting of the one or more blood samples. In some embodiments, the subject was bitten by a tick carrying Borrelia bacteria at least a year prior to the collecting of the one or more blood samples. In some embodiments, the subject has arthritis, and a cause of the arthritis has not been determined prior to the collecting of the one or more blood samples. In some embodiments, the subject is serologically positive for Borrelia antibodies. In some embodiments, a concentration of Borrelia mcfDNA comprises 1-100 molecules per microliter (MPM) of plasma. In some embodiments, a concentration of Borrelia mcfDNA comprises 1-1,000 molecules per microliter (MPM) of plasma. In some embodiments, the subject has disseminated late-stage Lyme disease. In some embodiments, a sensitivity of detecting the Borrelia mcfDNA is at least 60%. In some embodiments, a sensitivity of detecting the Borrelia mcfDNA is at least 60%, at least 65%, at least 70%, at least 75%, at least 80%, at least 85%, at least 90%, or at least 95%. In some embodiments, a Borrelia mcfNA comprises mcfNA derived from B. burgdorferi or B. maynoii bacteria. In some embodiments, detecting Borrelia mcfNA comprises performing a sequencing-by-synthesis assay on the mcfNA. In some embodiments, the detecting the Borrelia mcfNA comprises performing a next-generation sequencing assay or a metagenomic sequencing assay on cell-free nucleic acids from the one or more blood samples. In some embodiments, the detecting the Borrelia mcfNA comprises performing a next-generation sequencing assay or a metagenomic sequencing assay on nucleic acids from the one or more blood samples.

Disclosed herein in some embodiments is a method of detecting or monitoring Borrelia spp. in a subject who has received a treatment for Lyme arthritis comprising (a) preparing a sample comprising cell-free nucleic acids comprising microbial cell-free nucleic acids (mcfNA) from the subject; (b) subjecting the cell-free nucleic acids to next generation sequencing to produce sequence reads; (c) aligning the sequence reads to genomic sequences from Borrelia spp. in a reference data set to obtain aligned sequence reads; (d) detecting a presence of and quantifying Borrelia spp. nucleic acids (e.g., DNA) based on the aligned sequence reads to obtain a threshold value; and (e) repeating (a) to (d) until Borrelia spp. nuclei acids (e.g., DNA) are below the threshold value, or are undetectable in the plasma. In some embodiments, mcfNA comprises microbial cell-free DNA. In some embodiments, a quantifying in (d) comprises detecting 1-100 molecule of Borrelia spp. DNA per microliter of plasma. In some embodiments, the subject is blood culture negative for Borrelia spp. bacteria during the collecting of the one or more blood samples. In some embodiments, the subject does not have erythema migrans rash. In some embodiments, the subject is at a high risk of having Lyme arthritis. In some embodiments, the subject has a geographic risk of having Lyme disease.

Disclosed herein in some embodiments is a method of treating a subject with Lyme arthritis comprising (a) preparing a sample comprising cell-free nucleic acids comprising microbial cell-free nucleic acids (mcfNA) from the subject; (b) subjecting the cell-free nucleic acids comprising mcfNA to next generation sequencing to produce sequence reads; (c) aligning the sequence reads to genomic sequences from Borrelia spp. in a reference data set to obtain aligned sequence reads; (d) detecting a presence of and quantifying Borrelia spp. nucleic acids (e.g., DNA) based on the aligned sequence reads; (e) administering a therapeutic treatment to the subject; and repeating (a) to (e) until Borrelia spp. nuclei acids (e.g., DNA) is undetectable in the plasma. In some embodiments, mcfNA comprises microbial cell-free DNA. In some embodiments, a quantifying in (d) comprises detecting 1-100 molecule of Borrelia spp. DNA per microliter of plasma. In some embodiments, the subject is blood culture negative for Borrelia spp. bacteria during the collecting of the one or more blood samples. In some embodiments, the subject does not have erythema migrans rash. In some embodiments, the subject is at a high risk of having Lyme arthritis. In some embodiments, the subject has a geographic risk of having Lyme disease.

Disclosed herein in an aspect is a method of detecting and treating Borrelia spp. in a subject, the method comprising: collecting one or more blood samples from the subject at a time when the subject has at least one erythema migrans (EM) rash and wherein the one or more blood samples comprise microbial cell-free nucleic acids (mcfNA); detecting mcfNA from Borrelia spp. in the one or more blood samples; and administering a treatment to the subject to treat an infection associated with the Borrelia spp. In some embodiments, the method comprises quantifying the mcfNA from Borrelia spp. In some embodiments, the mcfNA from Borrelia spp is microbial cell-free DNA from Borrelia spp. In some embodiments, one or more blood samples are one or more plasma samples. In some embodiments, the method further comprises attaching the mcfNA to nucleic acid adapters to prepare a sequencing library comprising the mcfNA. In some embodiments, the method further comprises generating sequence reads from the sequencing library comprising the mcfNA, aligning the sequence reads to Borrelia spp. genomic sequences in a reference data set to obtain aligned sequence reads, and identifying the Borrelia spp. based on the aligned sequence reads. In some embodiments, the treatment is a Borrelia-directed therapy. In some embodiments, the Borrelia-directed therapy is at least one therapy selected from the group consisting of: doxycycline, amoxicillin, cefuroxime axetil, ceftriaxone, and cefotaxime. In some embodiments, the method further comprises spiking the one or more blood samples with a known concentration of synthetic DNA. In some embodiments, the method comprises spiking the one or more plasma samples with a known concentration of synthetic DNA. In some embodiments, a concentration of the Borrelia mcfNA per microliter of blood is measured. In some embodiments, a concentration of Borrelia mcfNA per microliter of blood is greater than a threshold amount. In some embodiments, the subject has arthritis. In some embodiments, the subject has arthritis of a joint. In some embodiments, the joint comprises at least one joint selected from the group consisting of knee, elbow, temporomandibular joint, and hip. In some embodiments, the subject is blood culture negative for Borrelia at the time of the collecting of the one or more blood samples. In some embodiments, the subject is negative for Borrelia when measured by a polymerase chain reaction (PCR) test of a sample of blood from the subject. In some embodiment, the subject is negative for Borrelia when measured by a polymerase chain reaction (PCR) test of a sample of synovial fluid from the subject. In some embodiments, the subject was bitten by a tick carrying Borrelia bacteria at least 6 months prior to the collecting of the one or more blood samples. In some embodiments, the subject was bitten by a tick carrying Borrelia bacteria at least a year prior to the collecting of the one or more blood samples. In some embodiments, the subject has arthritis and a cause of the arthritis has not been determined prior to the collecting of the one or more blood samples. In some embodiments, the subject is serologically positive for Borrelia antibodies. In some embodiments, a concentration of Borrelia mcfDNA comprises 1-100 molecules per microliter (MPM) of plasma. In some embodiments, a concentration of Borrelia mcfDNA comprises 1-1,000 molecules per microliter (MPM) of plasma. In some embodiments, the subject has disseminated late-stage Lyme disease. In some embodiments, a sensitivity of detecting the Borrelia mcfDNA comprises at least 60%. In some embodiments, a Borrelia mcfNA comprises mcfNA derived from B. burgdorferi or B. maynoii bacteria. In some embodiments, a detecting the Borrelia mcfNA comprises performing a sequencing-by-synthesis assay on the mcfNA, e.g., by performing a sequencing-by-synthesis assay on cell-free nucleic acids comprising the mcfNA. In some embodiments, the subject has a single erythema migrans (EM) rash. In some embodiments, the subject has multiple erythema migrains (EM) rashes.

Disclosed herein in an aspect is a method of treating a subject with Lyme arthritis comprising (a) preparing a sample comprising cell-free nucleic acids comprising microbial cell-free nucleic acids (mcfNA) from the subject; (b) subjecting the cell-free nucleic acids comprising mcfNA to next generation sequencing to produce sequence reads; (c) aligning the sequence reads to genomic sequences from Borrelia spp. in a reference data set to obtain aligned sequence reads; (d) detecting a presence of and quantifying Borrelia spp. DNA based on the aligned sequence reads; (e) administering a therapeutic treatment to the subject; and (f) repeating (a) to (e) until Borrelia spp. DNA is undetectable in the plasma. In some embodiments, the mcfNA comprises microbial cell-free DNA. In some embodiments, a quantifying in (d) comprises detecting 1-100 molecules of Borrelia sppsingl. DNA per microliter of plasma. In some embodiments, the subject is blood culture negative for Borrelia spp. bacteria during the collecting of the one or more blood samples. In some embodiments, the subject does not have at least one erythema migrans rash.

Disclosed herein in an aspect is a method of detecting Borrelia spp. in a subject, the method comprising: collecting one or more blood samples from the subject at a time when the subject has at least one erythema migrans (EM) rash and wherein the one or more blood samples comprise microbial cell-free nucleic acids (mcfNA); preparing a sequencing library by attaching nucleic acid adapters to the mcfNA such as by attaching adapters to cell-free nucleic acids in or from the one or more blood samples; subjecting the mcfNA to next-generation sequencing to obtain sequence reads; aligning the sequence reads to a reference genome comprising sequences from Borrelia spp. to obtain aligned sequence reads; and detecting mcfNA from Borrelia spp. based on the aligned sequence reads. In some embodiments, the method further comprises quantifying the mcfNA from Borrelia spp. In some embodiments, the mcfNA from Borrelia spp comprises microbial cell-free DNA from Borrelia spp. In some embodiments, one or more blood samples are one or more plasma samples. In some embodiments, the method further comprises spiking the one or more blood samples with a known concentration of synthetic DNA. In some embodiments, the method further comprises spiking the one or more plasma samples with a known concentration of synthetic DNA. In some embodiments, a concentration of the Borrelia mcfNA per microliter of blood is measured. In some embodiments, a concentration of Borrelia mcfNA per microliter of blood is greater than a threshold amount. In some embodiments, the subject has early localized Lyme disease. In some embodiments, the subject has early disseminated Lyme disease. In some embodiments, the subject has late-stage Lyme disease. In some embodiments, the subject has arthritis. In some embodiments, the subject has arthritis of a joint. In some embodiments, the joint comprises at least one joint selected from the group consisting of knee, elbow, temporomandibular joint, and hip. In some embodiments, the subject is negative for Borrelia when measured by a polymerase chain reaction (PCR) test of a sample of blood from the subject. In some embodiment, the subject is negative for Borrelia when measured by a polymerase chain reaction (PCR) test of a sample of synovial fluid from the subject. In some embodiments, the subject was bitten by a tick carrying Borrelia bacteria at least 6 months prior to the collecting of the one or more blood samples. In some embodiments, the subject was bitten by a tick carrying Borrelia bacteria at least a year prior to the collecting of the one or more blood samples. In some embodiments, the subject has arthritis and a cause of the arthritis has not been determined prior to the collecting of the one or more blood samples. In some embodiments, the subject is serologically positive for Borrelia antibodies. In some embodiments, a concentration of Borrelia mcfDNA comprises 1-100 molecules per microliter (MPM) of plasma. In some embodiments, a concentration of Borrelia mcfDNA comprises 1-1,000 molecules per microliter (MPM) of plasma. In some embodiments, the subject has disseminated late-stage Lyme disease. In some embodiments, a sensitivity of detecting the Borrelia mcfDNA comprises at least 60%. In some embodiments, a Borrelia mcfNA comprises mcfNA derived from B. burgdorferi or B. maynoii bacteria. In some embodiments, detecting the Borrelia mcfNA comprises performing a sequencing-by-synthesis assay on the mcfNA. In some embodiments, the subject has a single erythema migrans (EM) rash. In some embodiments, the subject has multiple erythema migrains (EM) rashes.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 provides a basic depiction of many of the methods provided herein.

FIG. 2 depicts an exemplary method of monitoring a response to a treatment for Lyme disease.

### DETAILED DISCLOSURE

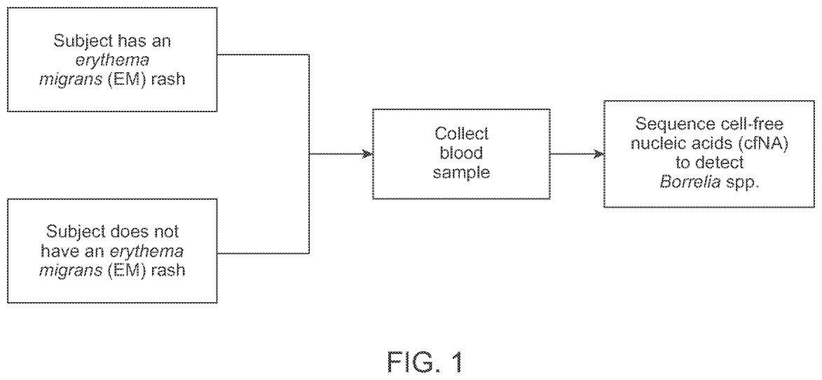

This disclosure provides methods of detecting or diagnosing a Lyme disease infection, particularly by detecting Borrelia bacteria. FIG. 1 provides a general depiction of many of the methods provided herein. In some cases, this disclosure provides methods of detecting or diagnosing a Lyme disease infection at a late stage of the infection. In some cases, the methods comprise detecting or diagnosing Lyme disease at time when the subject does not have an erythema migrans (EM) rash. In some cases, the subject previously had an EM rash, but the rash has cleared at the time of the collection of a body sample (e.g., blood sample, plasma sample) for use in the methods provided herein. In some cases, the subject has not had an EM rash in the past. In some cases, the methods comprise detecting or diagnosing Lyme disease at a time when the subject has arthritis (e.g., arthritis of a joint, an elbow, a knee, a hip, or a temporomandibular joint). In such cases, the methods may comprise detecting or diagnosing Lyme arthritis in the subject. For example, the subject may already be known to have arthritis, but the methods provided herein enable the determination that the arthritis is associated with Lyme disease. In some cases, such subject is known or suspected to have previously contracted Lyme disease; but often, it is not known whether Lyme disease is causing the arthritis. In some cases, the subject is serologically positive for Lyme disease. But even in such cases, it may not be clear that Lyme disease is causing the arthritis, given the persistence of antibody seropositivity over a long period of time. As such, the methods provided herein may be particularly valuable for subjects who were infected by Borrelia spp. several months prior to the collection of the sample from the subject, such as at least 4 months, 6 months, 12 months, 18 months, 24 months, 36 months prior to collection.

In some cases, the methods may comprise detecting or diagnosing Lyme disease in a subject at an early stage, such as an early localized stage or early disseminated stage of the disease. In such cases, the subject can have an erythema migrans (EM) rash (in the case of early localized disease) or multiple EM rashes (in the case of early disseminated disease). In some cases, this disclosure provides methods of detecting nucleic acids (e.g., microbial cell-free nucleic acids (mcfNA), microbial cell-free DNA (mcfDNA)) from Borrelia spp., the causative agent of Lyme disease. Borrelia bacteria are a type of spirochete, a gram-negative bacterium with a spiral or corkscrew shape. In some cases, the methods comprise detecting mcfNA or mcfDNA derived from Borrelia bacteria in a body fluid of a subject, particularly a blood, plasma, serum, urine, synovial fluid or saliva sample from a subject. The methods can further comprise treating the subject for an infection caused by or associated with the detected Borrelia bacteria. In some cases, the subject is blood culture negative for Borrelia bacteria. In some cases, the subject is negative for Borrelia bacteria when a polymerase chain reaction (PCR) test is conducted on sample from the subject such as a whole blood, plasma, synovial fluid, urine or serum sample from the subject. In still other cases, synovial fluid from the subject can be positive for Borrelia bacteria, either by culture or PCR. However, in some cases, synovial fluid from the subject is negative for Borrelia bacteria by culture or PCR.

In some cases, the subject was bitten by a tick carrying Borrelia bacteria at least 6 months or at least a year prior to collecting one or more blood samples. In some cases, when the subject has arthritis, a cause of the arthritis has not been determined prior to collect a blood sample. In some cases, the subject is serologically positive for Borrelia antibodies. In some cases, the subject has disseminated late-stage Lyme disease.

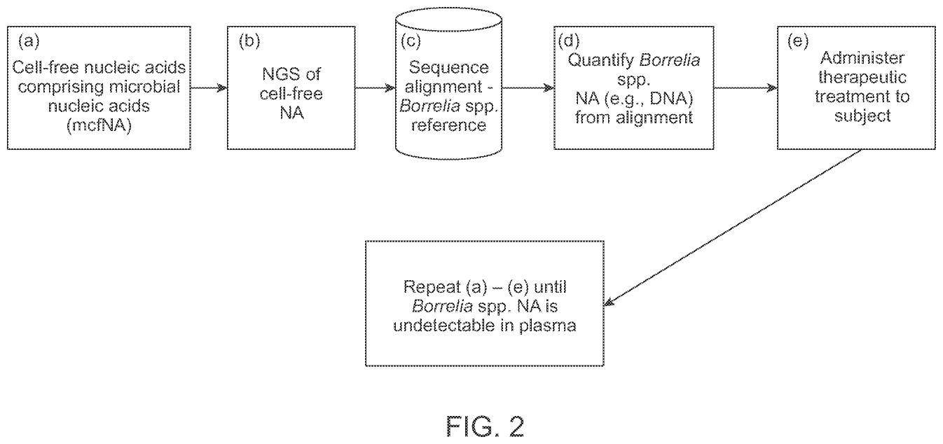

In some cases, the methods comprise methods for detecting mcfDNA in a subject (e.g., patient) to detect or monitor the subject's response to an antimicrobial treatment (e.g., antibiotic). An exemplary method is depicted in FIG. 2. In some instances, the subject is being treated for an infection such as a localized infection. For example, the infection is arthritis, particularly Lyme arthritis-which, in some cases, is negative for Borrelia bacteria by blood culture or by PCR of a blood sample from the subject. In some embodiments, the infection is a Borrelia spp. infection. In some cases, the infection is localized to an organ. In some cases, the infection is localized to a joint. In some cases, the infection is localized to an organ such as heart, mitral valve, lung, liver, kidney, cardiac tissue, cardiac sac, and/or aorta. The methods provided herein are particularly useful for fastidious or unculturable microbes (e.g., pathogens). Generally, the methods provided herein involve detection and/or quantification of microbial cell free nucleic acids (e.g., microbial cell-free DNA, microbial cell-free RNA) in a sample from a subject (e.g., plasma).

In some cases, this disclosure provides methods method of monitoring a treatment of a Borrelia spp. microbial infection in a subject comprising (a) preparing an initial sample (e.g., plasma) comprising microbial cell-free nucleic acids (mcfNA) from the subject, and, optionally, a known amount of a first synthetic nucleic acid (sNA); (b) measuring a threshold amount of mcfNA in the initial plasma sample; (c) preparing a longitudinal sample (e.g., plasma sample) comprising mcfNA and, optionally, a known amount of a second sNA (e.g., synthetic nucleic acid); (d) measuring a second mcfNA concentration in the longitudinal plasma sample relative to the second sNA; and (e) repeating (c) and (d) and maintaining the treatment until the mcfNA concentration in the longitudinal blood sample is significantly lower than the threshold mcfNA concentration. In some cases, this disclosure provides a method of treating a microbial infection in a subject comprising (a) preparing an initial plasma sample comprising microbial cell-free nucleic acids (mcfNA); (b) measuring a threshold concentration of mcfNA in the initial plasma sample; (c) treating the subject for the microbial infection; (d) preparing a longitudinal plasma sample comprising mcfNA; (e) measuring a second mcfNA concentration in the longitudinal plasma sample; (f) treating the subject for the microbial infection when the second mcfNA concentration is substantially greater than the threshold mcfNA concentration; and (g) repeating (c)-(f) until the mcfNA concentration in a longitudinal blood sample is significantly lower than the threshold mcfNA concentration, or preferably when the level of microbial mcfDNA becomes undetectable (0 MPM).

In some cases, the method is a method of detecting a Borrelia spp. microbial infection in a subject comprising (a) preparing an initial plasma sample comprising microbial cell-free nucleic acids (mcfNA); (b) analyzing mcfNA to identify the microbial infection at a species or strain level; (c) measuring a threshold concentration of mcfNA in the initial plasma sample relative to the sNA; (d) preparing a longitudinal plasma sample comprising the mcfNA and a known amount of a second sNA; (e) measuring a second mcfNA concentration in the longitudinal plasma sample relative to the second sNA; and (f) repeating (d) and (e) until the mcfNA concentration in a longitudinal blood sample is significantly lower than the threshold mcfNA concentration.

The methods can comprise attaching a nucleic acid adapter (e.g., DNA adapter) to the cell-free nucleic acids (e.g., cell-free DNA) and preparing a sequencing library. In some cases, the methods comprise attaching a first adapter to DNA from a first subject and a second adapter comprising a different sequence to a DNA sample from a second subject to produce first and second DNA libraries respectively. In some cases, the first and second DNA libraries are combined. The libraries may be subjected to multiplex sequencing (e.g., next generation sequencing, metagenomic sequencing), after which the sequence reads are demultiplexed. In some cases, samples (or libraries derived therefrom) from multiple subjects (e.g., at least 2, 3, 4, 5, 7, 10 subjects) are combined during the process of multiplex sequencing. In some cases, the sequencing comprises performing sequencing-by-synthesis reactions using reversible terminators, particularly fluorescently labeled reversible terminators (e.g., fluorescently labeled ddNTP, dNTP). In some embodiments, sequence reads exhibiting strong alignment against human references or the synthetic molecule references are excluded from the analysis. In some cases, sequence reads are filtered based on sequencing quality. In some embodiments, the remaining reads are aligned against a microbe database. In some embodiments, an expectation maximization algorithm is applied to compute the maximum likelihood estimate of each taxon abundance. In some cases, quantity of each microbe is expressed as Molecules per Microliter (MPM), which can refer to the number of DNA sequence reads from the reported microbe (e.g., bacterium, fungus, virus) present per microliter of plasma, or other biological fluid. In some embodiments, the method further comprises treating the subject for the infection, such as by administering a treatment, maintaining a treatment, or adjusting a dose of treatment. In some cases, the treatment is an antimicrobial treatment (e.g., antibiotic, or antifungal drug). In some cases, the treatment is a broad-spectrum drug. In some cases, the treatment specifically targets a particular microbe.

In some cases, the sample (e.g., plasma sample) is spiked with a known concentration of synthetic DNA for quality control purposes. In some cases, the method further comprises performing next generation sequencing on the synthetic DNA (or other sNA) in order to determine if there has a been a loss of synthetic DNA or sNA following sample processing. In some cases, such loss can be used to adjust the concentration of the target nucleic acid, e.g., a nucleic acid associated with Borrelia.

The methods provided herein generally have the advantage of being rapid and non-invasive. In some cases, the process from DNA extraction to analysis is completed in at most 20 hours, at most 24 hours, at most 28 hours, at most 30 hours, at most 36 hours, or at most 48 hours.

Numeric ranges are inclusive of the numbers defining the range. The term “about” as used herein generally means plus or minus ten percent (10%) of a value, inclusive of the value, unless otherwise indicated by the context of the usage. For example, “about 100” refers to any number from 90 to 110.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

The term “attach” and its grammatical equivalents may refer to connecting two molecules using any mode of attachment. For example, attaching may refer to connecting two molecules by chemical bonds or other method to generate a new molecule. Attaching an adapter to a nucleic acid may refer to forming a chemical bond between the adapter and the nucleic acid. In some cases, attaching is performed by ligation, e.g., using a ligase. For example, a nucleic acid adapter may be attached to a target nucleic acid by ligation, via forming a phosphodiester bond catalyzed by a ligase. In some cases, an adapter can be attached to a target nucleic acid (or copy thereof) using a primer extension reaction.

As used herein, the term “or” is used to refer to a nonexclusive or, such as “A or B” includes “A but not B,” “B but not A,” and “A and B,” unless otherwise indicated.

As used herein, “a”, “an”, and “the” can include plural referents unless otherwise limited expressly or by context.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

### Subjects

The term “subject” as used herein includes patients, particularly human patients. The term “subject” also encompasses other mammals, laboratory animals, veterinary animals, dogs, cats, and rodents.

In some embodiments, a subject has an infection, particularly at the time of collection of a sample from the subject. In some embodiments, the subject has no sign of an infection. In some embodiments, the subject is blood-culture negative for Borrelia bacteria at the time of collection of a sample. In some embodiments, the subject is blood-culture positive at the time of collection of a sample. In some cases, culture of a site of infection of the subject, e.g., a biopsy tissue or a bodily fluid (e.g., synovial fluid) is negative at the time of collection of the sample. In some embodiments, the subject is blood-culture positive at the time of collection of a sample for one or more pathogens and blood culture negative for one or more pathogens that later develop into an infection. In some cases, the subject is blood culture negative for a microbe or pathogen detected by the methods provided herein at the time of collection of the sample. In some embodiments, a subject has symptoms of infection at the time of collection of a sample or samples from the subject. In some embodiments, a symptom of an infection includes a fever, chills, elevated temperature, fatigue, a cough, congestion, fever, elevated heart rate, low blood pressure, hyperventilation, a sore throat, or any combination thereof. In some embodiments, a fever is a rectal, ear or temporal artery temperature of 100.4° F. (38° C.) or higher, an oral temperature of 100° F. (37.8° C.) or higher, an armpit temperature of 99° F. (37.2° C.) or higher, or any combination thereof. In some cases, the subject has symptoms of infection relative to a specific organ, such as symptoms related to an infected brain, heart, kidney or other organ.

In some embodiments disclosed herein, a subject is at risk of having an infection (e.g., high risk of having an infection), particularly at the time of collecting a sample from the subject. As used herein, a subject with a “high risk” of experiencing an infection is a subject with a risk that is higher than that of a healthy subject. For example, a patient may be at “high risk” of having Lyme arthritis if the patient has previously had Lyme disease. In some cases, the subject has a geographic risk of infection. A subject with a geographic risk may, for example, be known to have visited an area known to have ticks carrying Borrelia bacteria.

In some embodiments, the subject is a child. In some embodiments, a child is less than about 18 years of age. In some embodiments, the subject is a pediatric subject. In some embodiments, a subject is an adult. In some embodiments, a subject is less than about 25 years of age. In some embodiments, a subject is elderly. In some embodiments a subject is more than 65 years of age. In some cases, the subject has a high risk of experiencing a bacterial or fungal infection.

In some embodiments, the subject has, is suspected of having, or is at risk (e.g., high risk) of having an infection by a bacterium, a fungus, a virus, a parasite, or any combination thereof, or has symptoms of such infection. In some embodiments, the infection is a fungal infection (e.g., invasive fungal infection). In some embodiments, the infection is a bacterial infection (e.g., localized infection). In some embodiments, a bacterial infection comprises an infection by a Borrelia spp. bacterium (e.g., B. burgdorferi, B. hermsii, B. mayonii, or B. miyamotoi).

Secondary infections by a microbe can also be detected. In some cases, the microbe is at least one fungus such as Aspergillus, Pneumocystis, Rhizopus, Cunninghamella, Mucor, Lichtheimia, or Rhizomucor. In some cases, the fungus is Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, or Rhizomucor meihei. In some cases, the microbe is a herpesvirus, e.g., a reactivating herpesvirus. In some embodiments the microbe or organism is at least one microbe or organism mentioned in the Examples section of this application. In some embodiments, the bacterial infection is a gram-negative bacterial infection. In some embodiments, the bacterial infection is a gram-positive bacterial infection. In some embodiments, the bacterial or fungal infection is susceptible to empirical antimicrobial therapy. In some embodiments, the subject is diagnosed with having an infection using methods disclosed herein.

In some cases, the subject has Lyme disease at a particular stage. Progression of Lyme disease generally follows three stages. In the first stage, known as the early localized stage, the infection has not yet spread throughout the body. The early localized stage can last for a few days to a few weeks after the initial tick bite. Often, the initial sign of Lyme infection is an erythema migrans (EM) rash at the site of the tick bite or localized swelling. An EM rash is generally an expanding rash that appears a few days or a few weeks (generally 3-32 days) after the bite. The rash often has a characteristic “bull's eye” pattern.” In some cases, the EM may grow to a size of 15 cm in diameter, or larger. The EM rash can be accompanied by symptoms of a viral-like illness such as fatigue, body aches, or headaches. The second stage is known as the early disseminated stage and occurs days or weeks after onset of the local infection. In the second stage, a subject can present with multiple EMs. The subject can also present with general symptoms such as fever, chills, fatigue, and lymphadenopathy or with symptoms associated with a particular organ such as the brain or heart (e.g., myocarditis). In early disseminated disease, Lyme disease can also impact the musculoskeletal system causing non-inflammatory transient arthritis and/or arthralgias. It can affect the nervous system manifesting as facial paralysis (Bell's palsy, classically bilateral), fatigue, and loss of memory. The third stage, known as the late disseminated stage or “late-stage Lyme disease”, occurs months to years after the initial infection. Patients at this stage can develop chronic symptoms that affect many parts of the body including the joints, central nervous system, brain, eyes, and heart. Generally, patients in the late stage of disease no longer have the EM rash. In some cases, Lyme arthritis starts six months after the initial infection. In some cases, the Lyme arthritis occurs greater than 6, 9, 10, 12, 16, 18, or 24 months following the initial infection. Lyme arthritis can impact one joint or multiple joints. Often, Lyme arthritis affects the knee. In some cases, Lyme arthritis affects a large joint (e.g., knee, hip). In some cases, Lyme arthritis affects a joint, an elbow, a knee, a hip, or a temporomandibular joint. In some cases, Lyme arthritis causes joint erosion. In some cases, the Lyme arthritis is not transient. In some cases, the Lyme arthritis is chronic.

In some cases, the subject has arthritis, particularly Lyme arthritis. In some cases, the arthritis is characterized by joint swelling, warmth, erythema, and/or limited range of motion. Often, one or more of the symptoms of arthritis have an acute onset. In some cases, the subject has arthritis with an unknown cause. In such cases, the methods provided herein can, in some instances, enable identification of the cause of the arthritis.

### Samples

In some embodiments, a sample is collected from a subject (e.g., a patient). In some embodiments, the sample is a biological sample. The samples analyzed in the methods provided herein are preferably any type of clinical sample. In some cases, the samples contain cells, tissue, or a bodily fluid. In preferred embodiments, the sample is a liquid or fluid sample. In some cases, the sample is a bodily fluid. In some cases, the sample is whole blood, plasma, serum, urine, stool, saliva, lymph, spinal fluid, synovial fluid, bronchoalveolar lavage, nasal swab, respiratory secretions, vaginal fluid, amniotic fluid, semen, or menses. In some cases, the sample is made up of, in whole or in part, cells or tissue. In some cases, cells, cell fragments, or exosomes are removed from the sample, such as by centrifugation or filtration.

In some embodiments, a biological sample is a whole blood sample. In some embodiments, the sample is a cell-free sample, such as a plasma sample or a cell-free plasma sample. In some embodiments, the sample is a sample of isolated or extracted nucleic acids (e.g., DNA, RNA, cell-free DNA). In some embodiments, the plasma sample is collected by collecting blood through venipuncture. In some embodiments, a specimen is mixed with an additive immediately after collection. In some cases, the additive is an anti-coagulant. In some cases, the additive prevents degradation of nucleic acids. In some cases, the additive is EDTA. In some embodiments, measures can be taken to avoid hemolysis or lipemia. In some embodiments, a sample is processed or unprocessed. In some embodiments, a sample is processed by extracting nucleic acids from a biological sample. In some embodiments, DNA is extracted from a sample. In some embodiments, nucleic acids are not extracted from the sample. In some embodiments, a sample comprises nucleic acids. In some embodiments, a sample consists essentially of nucleic acids.

In some cases, the methods provided herein comprise processing whole blood into a plasma sample. In some embodiments, such processing comprises centrifuging the whole blood to separate the plasma from blood cells. In some cases, the method further comprises subjecting the plasma to a second centrifugation, often at a higher speed to remove bacterial cells and cellular debris. In some cases, the second centrifugation is at a relative centrifugal force (rcf) of least about 4,000 rcf, at least about 5,000 rcf, at least about 6,000 rcf, at least about 8,000 rcf, at least about 10,000 rcf, at least about 12,000 rcf, at least about 14,000 rcf, at least about 16,000 rcf, or at least about 20,000 rcf.

In some cases, the method comprises collecting, obtaining, or providing a sample. In some cases, the method comprises collecting, obtaining, or providing multiple samples, e.g., multiple samples from the subject or patient. In some embodiments, the sample is collected when the subject has an infection. In some cases, the sample is collected prior the subject having an infection. In some cases, the sample is collected while the subject is receiving treatment for an infection. In some cases, the sample is collected after the subject has received a treatment for an infection. In some cases, additional samples are collected from the subject over time. In some embodiments, a second sample is collected from the subject at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days after the collection of an initial (or other) sample from the subject

In some cases, the sample is obtained at least 1, 2, 3, 4, 5, 6, 7, 8, 9, 10, 12, 14, 16, 18, 20, 22, 24, 26, 28, 30, 32, 34, 36, 38, 40, 42, 44, 46, 48 months, or longer after the subject is initially infected, e.g., by being bitten by a tick. In some cases, the sample is obtained less than 1, 2, 3, 4, 5, 6, or 12 months, after the subject is initially infected, e.g., by being bitten by a tick. In some cases, the sample is obtained less than 1, 2, 3, 4, 5, 6, or 12 weeks, after the subject is initially infected, e.g., by being bitten by a tick.

In some embodiments, a plurality of samples is collected over a series of time points. In some embodiments, a plurality of samples is collected to monitor an onset of a disease, to monitor progression of a disease, to detect a response to treatment for the disease or any combination thereof. In some embodiments, the plurality of samples is at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected before onset of a symptom. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected over a period of time. In some embodiments, a plurality of samples is collected on consecutive days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected on consecutive days. In some embodiments, a plurality of samples is collected on alternate days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples can be collected on alternate days. In some embodiments, the collection of samples can be interspersed between days when no sample is collected. In some embodiments, a schedule of sample collection can repeat over several days. In some embodiments, a schedule of sample collection can repeat over 2 days, over 3 days, over 4 days, over 5 days, over 6 days, over 7 days, over 8 days, over 9 days, over 10 days, over 11 days, over 12 days, over 13 days, over 14 days, over 15 days, over 16 days, over 17 days, over 18 days, over 19 days, over 20 days, over 21 days, or over 22 days. In some embodiments, a schedule of sample collection can repeat on the same day, collecting multiple samples from a subject throughout the 24 hours.

Often, a sample disclosed herein comprises a target nucleic acid (e.g., target DNA, target RNA). In some embodiments, a target nucleic acid is a cell-free nucleic acid. For example, the sample can comprise microbial cell-free nucleic acids (e.g., mcfDNA) that comprises a microbial target DNA (e.g., mcfDNA derived from a microbe, which can include pathogenic microbes). Exemplary microbes that can be detected by the methods provided herein include bacteria, fungi, parasites, and viruses. In some embodiments, a cell-free nucleic acid is a circulating cell-free nucleic acid. In some embodiments, a cell free nucleic acid can comprise cell-free DNA.

In some embodiments, nucleic acids (e.g., cell-free nucleic acids) are extracted from a sample. In still other embodiments, nucleic acids (e.g., cell-free nucleic acids) are not extracted from the sample prior to preparation of a sequencing library. In some embodiments, isolated nucleic acids (e.g., extracted DNA, extracted RNA) can be used to prepare DNA libraries. In some embodiments, DNA libraries can be prepared by attaching adapters to nucleic acids. In some embodiments, adapters can be used for sequencing of nucleic acids. In some embodiments, nucleic acids can comprise DNA. In some embodiments, nucleic acids containing adapters can be sequenced to obtain sequence reads. In some embodiments, a sample (e.g., a plasma sample comprising mcfDNA) is mixed with adapters prior to extracting nucleic acids or DNA from the sample. In some embodiments, nucleic acids extracted from a sample (e.g., a plasma sample comprising mcfDNA) are attached to adapters following extraction. In some embodiments, sequence reads can be produced through high-throughput sequencing (HTS). In some embodiments, HTS can comprise next-generation sequencing (NGS). In some embodiments, sequence reads can be aligned to sequences in a reference dataset. In some embodiments, sequences can be a bacterial sequence aligned to a reference dataset to obtain an aligned sequence read. In some embodiments, a sequence can be a fungal sequence aligned to a reference dataset to obtain an aligned sequence read. In some embodiments, an aligned bacterial sequence, a fungal sequence, or a combination thereof, can be quantified for bacterial sequences or fungal sequences based on aligned sequence reads obtained.

In the methods provided herein, nucleic acids can be isolated. In some embodiments, nucleic acids can be extracted using a liquid extraction. In some embodiments, a liquid extraction can comprise a phenol-chloroform extraction. In some embodiments, a phenol-chloroform extraction can comprise use of TRIZOL™, DNAZOL™, or any combination thereof. In some embodiments, nucleic acids can be extracted using centrifugation through selective filters in a column. In some embodiments, nucleic acids can be concentrated or precipitated by known methods, including, by way of example only, centrifugation. In some embodiments, nucleic acids can be bound to a selective membrane (e.g., silica) for the purposes of purification. In some embodiments, nucleic acids can be extracted using commercially available kits (e.g., QIAamp CIRCULATING NUCLEIC ACID KIT™, Qiagen DNeasy KIT™, QIAamp KIT™, Qiagen Midi KIT™, QIAprep SPIN KIT™, or any combination thereof). Nucleic acids can also be enriched for fragments of a desired length, e.g., fragments which are less than 1000, 500, 400, 300, 200 or 100 base pairs in length. In some embodiments, enrichment based on size can be performed using, e.g., PEG-induced precipitation, an electrophoretic gel or chromatography material (Huber et al. (1993) Nucleic Acids Res. 21:1061-6), gel filtration chromatography, or TSK gel (Kato et al. (1984) J. Biochem, 95:83-86), which publications are hereby incorporated by reference in their entireties for all purposes.

In some embodiments, a nucleic acid sample can be enriched for a target nucleic acid. In some embodiments, a target nucleic acid is a microbial cell-free nucleic acid.

In some embodiments, target (e.g., pathogen, microbial) nucleic acids are enriched relative to background (e.g., subject) nucleic acids in a sample, for example, by electrophoresis, gel electrophoresis, pull-down (e.g., preferentially pulling down target nucleic acids in a pull-down assay by hybridizing them to complementary oligonucleotides conjugated to a label such as a biotin tag and using, for example, avidin or streptavidin attached to a solid support), targeted PCR, or other methods. Examples of enrichment techniques include but are not limited to: (a) self-hybridization techniques in which a major population in a sample of nucleic acids self-hybridizes more rapidly than a minor population in a sample; (b) depletion of nucleosome-associated DNA from free DNA; (c) removing and/or isolating DNA of specific length intervals; (d) exosome depletion or enrichment; and (e) strategic capture of regions of interest.

In some embodiments, an enriching step can comprise preferentially removing nucleic acids from a sample that are above about 120, about 150, about 200, or about 250 bases in length. In some embodiments, an enriching step comprises preferentially enriching nucleic acids from a sample that are between about 10 bases and about 60 bases in length, between about 10 bases and about 120 bases in length, between about 10 bases and about 150 bases in length, between about 10 bases and about 300 bases in length between about 30 bases and about 60 bases in length, between about 30 bases and about 120 bases in length, between about 30 bases and about 150 bases in length, between about 30 bases and about 200 bases in length, or between about 30 bases and about 300 bases in length. In some embodiments, an enriching step comprises preferentially digesting nucleic acids derived from the host (e.g., subject). In some embodiments, an enriching step comprises preferentially replicating the non-host nucleic acids.

In some embodiments, a nucleic acid library is prepared. In some embodiments, a double-stranded DNA library, a single-stranded DNA library or an RNA library is prepared. A method of preparing a dsDNA library can comprise ligating an adaptor sequence onto one or both ends of a dsDNA fragment. In some cases, the adaptor sequence comprises a primer docking sequence. In some cases, the method further comprises hybridizing a primer to the primer docking sequence and initiating amplification or sequencing of the nucleic acid attached to the adaptor. In some embodiments, the primer or the primer docking sequence comprises at least a portion of an adaptor sequence that couples to a next-generation sequencing platform. In some embodiments, a method can further comprise extension of a hybridized primer to create a duplex, wherein a duplex comprises an original ssDNA fragment and an extended primer strand. In some embodiments, an extended primer strand can be separated from an original ssDNA fragment. In some embodiments, an extended primer strand can be collected, wherein an extended primer strand is a member of an ssDNA library.

In some cases, the library is prepared in an unbiased manner. For example, in some cases, the library is prepared without using a primer that specifically hybridizes to a microbial nucleic acid based on a predetermined sequence of the microbe. For example, in some embodiments, the only amplification performed on the sample involves the use of a primer specific for a sequence of one or more adapters attached to nucleic acids within the sample. In some cases, whole genome amplification is used to prepare the library prior to attachment of the adapters. In some cases, whole genome amplification is not used to prepare the library. In some cases, one or more primers that specifically hybridize to a microbial nucleic acid (e.g., pathogen, viral, fungal, bacterial or parasite nucleic acid) are used to amplify the sample.

In some cases, multiple DNA libraries from different samples (e.g., samples from different patients or subjects) are combined and then subjected to a next generation sequencing assay. In some cases, the libraries are indexed prior to combining to track which library corresponds to which sample. Indexing can involve the inclusion of a specific code or bar code in an adapter, e.g., an adapter that is attached to the nucleic acids are to be analyzed. In some cases, the samples comprise a negative control sample or a positive control sample, or both a negative control sample and a positive control sample.

In some embodiments, a length of a nucleic acid can vary. In some embodiments, a nucleic acid or nucleic acid fragment (e.g., dsDNA fragment, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. In some embodiments, a DNA fragment can be about 40 to about 100 bp, about 50 to about 125 bp, about 100 to about 200 bp, about 150 to about 400 bp, about 300 to about 500 bp, about 100 to about 500 bp, about 400 to about 700 bp, about 500 to about 800 bp, about 700 to about 900 bp, about 800 to about 1000 bp, or about 100 to about 1000 bp. In some embodiments, a nucleic acid or nucleic acid fragment (e.g., dsDNA fragment, RNA, or randomly sized cDNA) can be within a range from about 20 to about 200 bp, such as within a range from about 40 to about 100 bp.

In some embodiments, an end of a dsDNA fragment can be polished (e.g., blunt-ended) or be subject to end-repair to create a blunt end. In some embodiments, an end of a DNA fragment can be polished by treatment with a polymerase. In some embodiments, a polishing can involve removal of a 3′ overhang, a fill-in of a 5′ overhang, or a combination thereof. In some embodiments, a polymerase can be a proof-reading polymerase (e.g., comprising 3′ to 5′ exonuclease activity). In some embodiments, a proofreading polymerase can be, e.g., a T4 DNA polymerase, Pol 1 Klenow fragment, or Pfu polymerase. In some embodiments, a polishing can comprise removal of damaged nucleotides (e.g., abasic sites).

In some embodiments, a ligation of an adaptor to a 3′ end of a nucleic acid fragment can comprise formation of a bond between a 3′ OH group of the fragment and a 5′ phosphate of the adaptor. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all 5′ phosphate groups are removed from nucleic acid fragments. In some embodiments, substantially all 5′ phosphates are removed from at least 50%, 55%, 60%, 65%, 70%, 75%, 80%, 85%, 90%, 95%, or greater than 95% of nucleic acid fragments in a sample. Removal of 5′ phosphate groups from a nucleic acid sample can be by any means known in the art. Removal of phosphate groups can comprise treating the sample with heat-labile phosphatase. In some embodiments, 5′ phosphate groups are not removed from the nucleic acid sample. In some embodiments, ligation of an adaptor to the 5′ end of the nucleic acid fragment is performed.

### Exemplary Sample Processing

What follows are non-limiting examples of methods provided by this disclosure. In some cases, plasma is spiked with a known concentration of synthetic normalization molecule controls. In some cases, the plasma is then subjected to cell-free NA (cfNA) extraction (e.g., extraction of cell-free DNA). The extracted cfNA can be processed by end-repair and ligated to adapters containing specific indexes to end-repaired cfDNA. The products of the ligation can be purified by beads. In some embodiments, the cfDNA ligated to adapters can be amplified with P5 and P7 primers, and the amplified, adapted cfDNA is purified.

Purified cfDNA attached to adapters derived from a plasma sample can be incorporated into a DNA sequencing library. Sequencing libraries from several plasma samples can be pooled with control samples, purified, and, in some embodiments, sequenced on Illumina sequencers using a 75-cycle single-end, dual index sequencing kit. Primary sequencing output can be demultiplexed followed by quality trimming of the reads. In some embodiments, the reads that pass quality filters are aligned against human and synthetic references and then excluded from the analysis, or otherwise set aside. Reads potentially representing human satellite DNA can also filtered, e.g., via a k-mer-based method; then the remaining reads can be aligned with a microbe reference database, (e.g., a database with 20,963 assemblies of high-quality genomic references). In some embodiments, reads with alignments that exhibit both high percent identity and/or high query coverage can be retained, except, e.g., for reads that are aligned with any mitochondrial or plasmid reference sequences. PCR duplicates can be removed based on their alignments. Relative abundances can be assigned to each taxon in a sample based on the sequencing reads and their alignments.

For each combination of read and taxon, a read sequence probability can be defined that accounts for the divergence between the microbe present in the sample and the reference assemblies in the database. A mixture model can be used to assign a likelihood to the complete collection of sequencing reads that included the read sequence probabilities and the (unobserved) abundances of each taxon in the sample. In some cases, an expectation-maximization algorithm is applied to compute the maximum likelihood estimate of each taxon abundance. From these abundances, the number of reads arising from each taxon can be aggregated up the taxonomic tree. The estimated taxa abundances from the no template control (NTC) samples within the batch can be combined to parameterize a model of read abundance arising from the environment with variations driven by counting noise. Statistical significance values can then be computed for each estimate of taxon abundance in each patient sample. In some embodiments, taxa that exhibit a high significance level, and are one of the 1449 taxa within the reportable range, comprise the candidate calls. Final calls can be made after additional filtering is applied, which accounts for read location uniformity as well as cross-reactivity risk originating from higher abundance calls. The microbe calls that pass these filters are reported along with abundances in MPM, as estimated using the ratio between the unique reads for the taxon and the number of observed unique reads of normalization molecules.

The amount of mcfDNA plasma concentration in each sample can then be quantified by using the measured relative abundance of the synthetic molecules initially spiked in the plasma.

### Analysis

Disclosed herein in some embodiments, are methods of analyzing nucleic acids. Such analytical methods include sequencing the nucleic acids as well as bioinformatic analysis of the sequencing results (e.g., sequence reads).

In some embodiments, a sequencing is performed using a next generation sequencing assay. As used herein, the term “next generation” generally refers to any high-throughput sequencing approach including, but not limited to one or more of the following: massively-parallel signature sequencing, pyrosequencing (e.g., using a Roche 454 GENOME ANALYZER™ sequencing device), ILLUMINA™ (SOLEXA™) sequencing (e.g., using an ILLUMINA™ NEXTSEQ™ 500), sequencing by synthesis (ILLUMINA™), ion semiconductor sequencing (ION TORRENT™), sequencing by ligation (e.g., SOLID™ sequencing), single molecule real-time (SMRT) sequencing (e.g., PACIFIC BIOSCIENCE™), polony sequencing, DNA nanoball sequencing (COMPLETE GENOMICS™), heliscope single molecule sequencing (HELICOS BIOSCIENCES™), metagenomic sequencing and nanopore sequencing (e.g., OXFORD NANOPORE™). In some embodiments, a sequencing assay can comprise nanopore sequencing. In some embodiments, a sequencing assay can include some form of Sanger sequencing. In some embodiments, a sequencing can involve shotgun sequencing; in some embodiments, a sequencing can include bridge amplification PCR.

In some embodiments, a sequencing assay comprises a Gilbert sequencing method. In some embodiments, a Gilbert sequencing method can comprise chemically modifying nucleic acids (e.g., DNA) and then cleaving them at specific bases. In some embodiments, a sequencing assay can comprise dideoxynucleotide chain termination or Sanger-sequencing.

In some embodiments, a sequencing-by-synthesis approach is used in the methods provided herein. In some embodiments, fluorescently labeled reversible-terminator nucleotides are introduced to clonally amplified DNA templates immobilized on the surface of a glass flowcell. During each sequencing cycle, a single labeled deoxynucleoside triphosphate (dNTP) may be added to the nucleic acid chain. The labeled terminator nucleotide may be imaged when added to identify the base and then the terminator group May be enzymatically cleaved to allow synthesis of the strand to proceed. A terminator group can comprise a 3′-O-blocked reversible terminator or a 3′-unblocked reversible terminator. Since all four reversible terminator-bound dNTPs (e.g., A, C, T, G) are generally present as single, separate molecules, natural competition may minimize incorporation bias.

In some embodiments, a method called Single-molecule real-time (SMRT) is used. In such approach, nucleic acids (e.g., DNA) are synthesized in zero-mode waveguides (ZMWs), which are small well-like containers with capturing tools located at the bottom of the well. The sequencing is performed with use of unmodified polymerase (attached to the ZMW bottom) and fluorescently labelled nucleotides flowing freely in the solution. The fluorescent label is detached from the nucleotide upon its incorporation into the DNA strand, leaving an unmodified DNA strand. A detector such as a camera may then be used to detect the light emissions; and the data may be analyzed bioinformatically to obtain sequence information.

In some embodiments, a sequencing by ligation approach is used to sequence the nucleic acids in a sample. One example is the next generation sequencing method of SOLID™ (Sequencing by Oligonucleotide Ligation and Detection) sequencing (Life Technologies). This next generation technology may generate hundreds of millions to billions of small sequence-reads at one time. The sequencing method may comprise preparing a library of DNA fragments from the sample to be sequenced. In some embodiments, the library is used to prepare clonal bead populations in which only one species of fragment is present on the surface of each bead (e.g., magnetic bead). The fragments attached to the magnetic beads may have a universal P1 adapter sequence attached so that the starting sequence of every fragment is both known and identical. In some embodiments, the method may further involve PCR or emulsion PCR. For example, the emulsion PCR may involve the use of microreactors containing reagents for PCR. The resulting PCR products attached to the beads may then be covalently bound to a glass slide. A sequencing assay such as a SOLID™ sequencing assay or other sequencing by ligation assay may include a step involving the use of primers. Primers may hybridize to the P1 adapter sequence or other sequence within the library template. The method may further involve introducing four fluorescently labelled di-base probes that compete for ligation to the sequencing primer. Specificity of the di-base probe may be achieved by interrogating every first and second base in each ligation reaction. Multiple cycles of ligation, detection and cleavage may be performed with the number of cycles determining the eventual read length. In some embodiments, following a series of ligation cycles, the extension product can be removed, and the template can be reset with a primer complementary to the n−1 position for a second round of ligation cycles. Multiple rounds (e.g., 5 rounds) of primer reset may be completed for each sequence tag. Through the primer reset process, each base may be interrogated in two independent ligation reactions by two different primers. For example, a base at read position 5 can be assayed by primer number 2 in ligation cycle 2 and by primer number 3 in ligation cycle 1.

In some embodiments, a detection or quantification analysis of oligonucleotides can be accomplished by sequencing. In some embodiments, entire synthesized oligonucleotides can be detected via full sequencing of all oligonucleotides by e.g., ILLUMINA™ HISEQ 2500™, including the sequencing methods described herein.

In some embodiments, the sequencing is accomplished through Sanger sequencing methods. Sequencing can also be accomplished using high-throughput systems some of which allow detection of a sequenced nucleotide immediately after or upon its incorporation into a growing strand, e.g., detection of sequence in real time or substantially real time. In some embodiments, high throughput sequencing generates at least 1,000, at least 5,000, at least 10,000, at least 20,000, at least 30,000, at least 40,000, at least 50,000, at least 100,000, or at least 500,000 sequence reads per hour. In some embodiments, each read is at least 50, at least 60, at least 70, at least 80, at least 90, at least 100, at least 120, or at least 150 bases per read. In some embodiments, each read is up to 2000, up to 1000, up to 900, up to 800, up to 700, up to 600, up to 500, up to 400, up to 300, up to 200, or up to 100 bases per read. Long read sequencing can include sequencing that provides a contiguous sequence read of longer than 500 bases, longer than 800 bases, longer than 1000 bases, longer than 1500 bases, longer than 2000 bases, longer than 3000 bases, or longer than 4500 bases per read.

In some embodiments, a high-throughput sequencing can involve the use of technology available by ILLUMINA™ GENOME ANALYZER IIX™, MISEQ PERSONAL SEQUENCER™, or HISEQ™ systems, such as those using HISEQ 2500™, HISEQ 1500™, HISEQ 2000™, or HISEQ 1000™. These machines use reversible terminator-based sequencing by synthesis chemistry. These machines can sequence 200 billion or more reads in eight days. Smaller systems may be utilized for runs within 3, 2, or 1 days or less time. Short synthesis cycles may be used to minimize the time it takes to obtain sequencing results.

In some embodiments, a high-throughput sequencing involves the use of technology available by ABI Solid System. This genetic analysis platform can enable massively parallel sequencing of clonally amplified DNA fragments linked to beads. The sequencing methodology is based on sequential ligation with dye-labeled oligonucleotides.

In some embodiments, a next-generation sequencing can comprise ion semiconductor sequencing (e.g., using technology from LIFE TECHNOLOGIES™ (ION TORRENT™)) Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA, an H+ ion can be released, which can be measured as a change in pH. The H+ ion can be converted to voltage and recorded by the semiconductor sensor. An array chip can be sequentially flooded with one nucleotide after another. In some embodiments, no scanning, light, or cameras are required. In some embodiments, an IONPROTON™ Sequencer is used to sequence nucleic acid. In some embodiments, an IONPGM™ Sequencer is used. The ION TORRENT PERSONAL GENOME MACHINE™ (PGM) can sequence 10 million reads in two hours.

In some embodiments, a high-throughput sequencing involves the use of technology available by HELICOS BIOSCIENCES™ Corporation (Cambridge, Massachusetts) such as the Single Molecule Sequencing by Synthesis (SMSS) method. SMSS can allow for sequencing the entire human genome in up to 24 hours. In some embodiments, SMSS may not require a pre amplification step prior to hybridization. In some embodiments, SMSS may not require any amplification. In some embodiments, methods of using SMSS are described in part in US Publication application Nos. 20060024711; 20060024678; 20060012793; 20060012784; and 20050100932, each of which are herein incorporated by reference.

In some embodiments, a high-throughput sequencing involves the use of technology available by 454 LIFESCIENCES™, Inc. (Branford, Connecticut) such as the PICO TITER PLATE™ device which includes a fiber optic plate that transmits chemiluminescent signal generated by the sequencing reaction to be recorded by a charge-coupled device (CCD) camera in the instrument. This use of fiber optics can allow for the detection of a minimum of 20 million base pairs in 4.5 hours. In some embodiments, methods for using bead amplification followed by fiber optics detection are described in Marguiles, M., et al. “Genome sequencing in microfabricated high-density picolitre reactors”, Nature, doi: 10.1038/nature03959; which is herein incorporated by reference.

In some embodiments, high-throughput sequencing is performed using Clonal Single Molecule Array (SOLEXA™, Inc.) or sequencing-by-synthesis (SBS) utilizing reversible terminator chemistry. Methods of using these technologies are described in part in U.S. Pat. Nos. 6,969,488; 6,897,023; 6,833,246; 6,787,308; and US Publication application Nos. 20040106110; 20030064398; 20030022207; and Constans, A., The Scientist 2003, 17 (13): 36, each of which are herein incorporated by reference.

In some embodiments, the next generation sequencing is nanopore sequencing. A nanopore can be a small hole, e.g., on the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from OXFORD NANOPORE TECHNOLOGIES™; e.g., a GRIDION™ system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene-based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, a nanopore sequencing technology from GENIA™ can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABSYS™. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM™ or Roche™. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by COMPLETE GENOMICS™; see e.g., Drmanac et al. (2010) Science 327:78-81, which is incorporated herein by reference). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize, and a single strand can fold onto itself to form a DNA nanoball (DNB™) which can be approximately 200-300 nanometers in diameter on average. A DNA nanoball can be attached (e.g., by adsorption) to a microarray (sequencing flow cell). The flow cell can be a silicon wafer coated with silicon dioxide, titanium and hexamethyldisilazane (HMDS) and a photo resistant material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

The methods provided herein may include use of a system that contains a nucleic acid sequencer (e.g., DNA sequencer and RNA sequencer) for generating DNA or RNA sequence information. The system may include a computer comprising software or code that performs bioinformatic analysis on the DNA or RNA sequence information. Bioinformatic analysis can include, without limitation, assembling sequence data, detecting, and quantifying genetic variants in a sample, including germline variants and somatic cell variants (e.g., a genetic variation associated with cancer or pre-cancerous condition, a genetic variation associated with infection, or a combination thereof). In some embodiments, the bioinformatic analysis determines the threshold value for an assay provided herein, such as a method of determining a response to treatment. In some cases, the bioinformatics analysis further compares the value obtained in a longitudinal sample against the threshold value to determine whether there is a response to treatment. In some cases, the threshold value is determined in terms of MPM. In some cases, the bioinformatics analysis applies a known threshold, such as a known threshold value for a particular condition or microbe.

Sequencing data may be used to determine genetic sequence information, ploidy states, the identity of one or more genetic variants, as well as a quantitative measures of the variants, including relative and absolute relative measures.

In some embodiments a sequencing can involve sequencing of a genome. In some embodiments a genome can be that of a microbe or pathogen as disclosed herein. In some embodiments, sequencing of a genome can involve whole genome sequencing or partial genome sequencing. In some embodiments, a sequencing can be unbiased and can involve sequencing all or substantially all (e.g., greater than 70%, 80%, 90%) of the nucleic acids in a sample. In some embodiments, a sequencing of a genome can be selective, e.g., directed to portions of a genome of interest. In some embodiments, sequencing of select genes, or portions of genes may suffice for a desired analysis. In some embodiments, polynucleotides mapping to specific loci in a genome can be isolated for sequencing by, for example, sequence capture or site-specific amplification.

In some embodiments disclosed herein, is a method comprising a process of analyzing, calculating, quantifying, or a combination thereof. In some embodiments, a method can be used to determine quantities of bacterial and fungal sequence reads. In some embodiments, metrics can be generated to determine quantities of bacterial sequences, fungal sequences, or a combination thereof.

In some embodiments, sensitivity of a test refers to a test's ability to correctly detect subjects with an infection who have an infection. In some embodiments, a sensitivity is a detection rate of a disease or infection. In some embodiments, a sensitivity is the proportion of people who test positive for a disease among those who have the disease. In some embodiments, a sensitivity can be calculated using the following formula: Sensitivity=(number of true positives)/(number of true positives+number of false negatives) or Sensitivity=(number of true positives)/(total number of sick individuals in a population); or Sensitivity=probability of a true positive. In some embodiments, the methods provided herein can detect Borrelia infection (e.g., Lyme arthritis, early-stage Lyme disease, late-stage Lyme disease) with a sensitivity of at least 50%, 60%, 70%, 75%, 85%, 90%, 95%, 99%, or more; and, in some instances, the sensitivity of the method is 100%. In some cases, the methods provided herein can detect Borrelia infection (e.g., Lyme arthritis, early-stage Lyme disease, late-stage Lyme disease) with a sensitivity from 60% to 100%, 70% to 95%, 70% to 100%, or 60% to 90%. In some cases, the methods provided herein can detect Borrelia infection (e.g., Lyme arthritis, early-stage Lyme disease, late-stage Lyme disease) with a sensitivity of at least 60%.

In some embodiments, a specificity can refer to a test's ability to correctly reject healthy subjects without an infection. In some embodiments, a specificity of a test can comprise a proportion of subjects who truly do not have an infection who test negative for the infection. In some embodiments, a specificity can be calculated using the following formula: Specificity=(number of true negatives)/(number of true negatives+number of false positives) or Specificity=(number of true negatives)/(total number of well individuals in a population); or Specificity=probability of a negative test when the patient is healthy or well. In some cases, specificity is the proportion of negative control samples for which no bacterial or fungal organisms were identified by mcfDNA sequencing. In some embodiments, the methods provided herein can detect Borrelia infection (e.g., Lyme arthritis, early-stage Lyme disease, late-stage Lyme disease) with a specificity of at least 50%, 60%, 70%, 75%, 85%, 90%, 95%, 99%, or more; and, in some instances, the specificity of the method is 100%. In some cases, the methods provided herein can detect Borrelia infection (e.g., Lyme arthritis, early-stage Lyme disease, late-stage Lyme disease) with a specificity from 60% to 100%, 70% to 95%, 70% to 100%, or 60% to 90%.

In some embodiments, the quantity of a microbe identified in a method provided herein is expressed in Molecules Per Microliter (MPM), the number of DNA sequencing reads from the reported microbe present per microliter of plasma. In some cases, detection of infection occurs when the MPM is greater than a threshold value. In some cases, such threshold value of MPM may be greater than 1, 2, 5, 6, 8, 10, 15, 20, 30, 50, 100, 200, 300, 400, 500, 600, 700, 800, 900, 1000, 2000, 5000, 7000, 10000, 20000, 30000, or 40000. In some cases, the MPM threshold is determined for a particular microbe.

In some embodiments, the quantity for a microbe (e.g., bacterium, fungus, virus) identified in a method provided herein is expressed as the amount or quantity of the microbe in a sample in relation to, or compared with, a threshold value, e.g., the amount of microbial cell-free nucleic acid in a sample as a percentage of the amount of the microbial cell-free nucleic acid in an initial sample. In some cases, the threshold value is an absolute value that can be used generally, irrespective of the subject. For example, the threshold value may be a normalized value signifying an average MPM value for a particular microbe in samples from a cohort of infected individuals prior to starting treatment for the infection. In some embodiments, the threshold value is the amount of a microbe measured in the initial sample (e.g., plasma, serum, cell-free sample) that is collected from the patient before beginning the treatment regimen for the microbial infection or while the patient is undergoing the treatment regimen for the microbial infection.

### EXAMPLES

The utility of metagenomic next-generation sequencing (mNGS) of plasma microbial cell-free DNA (mcfDNA) for the direct detection of B. burgdorferi among children and adults with untreated Lyme disease was measured herein. mcfDNA mNGS is a novel technology that provides unbiased identification of pathogens that may be otherwise difficult to diagnose. Unlike PCR, mcfDNA mNGS does not target a specific DNA sequence, potentially increasing the yield of positive results. The results support the conclusion that mNGS is capable of specifically detecting B. burgdorferi mcfDNA in the plasma of patients with untreated Lyme disease at various clinical stages.

These cohort studies were approved by the Institutional Review Boards at Stony Brook University Hospital and the Johns Hopkins University School of Medicine. At Stony Brook University Hospital in Suffolk County, NY pediatric patients aged 1-17 years old presenting with untreated Lyme disease at various clinical stages were enrolled. At the Johns Hopkins University Lyme Disease Research Center, participants over the age of 18 with early Lyme disease as well as non-Lyme infected control participants were enrolled.

Pediatric patients with presumed Lyme disease were defined as having one or more of the following: single or multiple EM (agreed upon by two pediatric infectious disease physicians, ASH and CB), unilateral or bilateral facial nerve palsy, carditis, meningitis (headache and/or meningismus, with pleocytosis >5 WBC/μL), and/or arthritis (acute-onset physician-documented joint swelling, warmth, erythema, and/or limited range of motion), without evidence of an alternative diagnosis. See Table 1 Pediatric cases were “confirmed” if serology was positive for Lyme disease by standard two-tier Lyme disease criteria. Early localized or early disseminated cases were “suspected” if they met clinical but not serologic criteria, and work-up revealed no alternative diagnosis. mNGS for mcfDNA was performed on all enrolled participants, even if an alternative diagnosis was made after enrollment. Pediatric participants were excluded if they had received oral or intravenous antibiotics within 30 days prior to enrollment, previously had Lyme disease, or had symptom resolution prior to enrollment and venipuncture. No sample size calculations were performed for this pilot study.

During the single study visit, a history was obtained using a standardized case report form, and physical examination and venipuncture were performed. For all participants, venipuncture occurred prior to or within 24 hours of receiving the first dose of antibiotics active against B. burgdorferi. Blood laboratory testing included standard serology (either VISE C6 peptide or whole-cell sonicate enzyme-linked immunosorbent assay (ELISA), both reflexed to Western blot), mNGS mcfDNA, and when possible, B. burgdorferi PCR. Other clinically obtained laboratory results were also recorded.

Adult participants were recruited from primary and urgent care settings, and all had an EM lesion ≥5 cm diagnosed by a physician present at the time of study enrollment (Table 2). A total of five participants were selected who were PCR/ESI-MS positive as well as two-tier antibody positive at either their acute or convalescent (end of treatment) study visit. Additionally, five participants who were PCR/ESI-MS negative were selected who were two-tier negative at both acute and convalescent time points. Non-Lyme infected control participants were recruited from clinic settings as well as online and paper flyers, did not have a clinical history of Lyme disease and were two-tier negative at study enrollment. Adult participants were excluded for prior Lyme disease or if they had received the Lyme disease vaccine. mNGS mcfDNA was performed on biobanked samples on all 15 participants.

Within 6 hours of venipuncture, whole blood samples from plasma-preparation tubes were processed to plasma and frozen at −20° C. or −80° C. McfDNA mNGS was performed in a blinded fashion to participant group by a Clinical Laboratory Improvement Amendments/College of American Pathologists-accredited laboratory as previously described. Briefly, mcfDNA was extracted from plasma followed by mNGS library preparation and sequencing. Human DNA reads were removed, and the remaining sequences were mapped to a pathogen genome database. Results were reported as molecules per microliter (MPM).

Thresholds for detection of statistically significant quantities of DNA were previously determined. Investigators were informed of all organisms detected above the commercial statistical threshold, and B. burgdorferi DNA detected at any level. Sub-threshold results were also reviewed for detection of other Borrelia species and additional pathogens transmitted by deer ticks, including B. hermsii, mayonii, and miyamotoi, Babesia microti, and Anaplasma phagocytophilum.

Fifteen adult participants in the validated cohort included ten participants with clinically suspected acute Lyme disease who presented with EM-five of whom had laboratory confirmed Lyme disease (Case Positive) and five of whom had no detectable serum antibody (Case Negative). See Table 2 Three of five confirmed cases presented with multiple/disseminated EM; one of five Case Negative participants presented multiple/disseminated EM. B. burgdorferi mcfDNA (14-173 MPM; mean 61+/−69) was detected in all the Case Positive participants with laboratory-confirmed Lyme disease (one reaching the commercial statistical threshold of the mcfDNA test disclosed herein) and in none of the Case Negative participants (0 MPM or the healthy asymptomatic control participants (0 MPM In an additional set of 684 asymptomatic healthy adults no participants had any B. burgdorferi mcfDNA detected, suggesting the specificity of B. burgdorferi mcfDNA when it is found in human plasma.

Fifteen pediatric participants with clinically suspected Lyme disease were enrolled in the real-world prospective cohort (Table 1). Participants presented with early localized disease (four with single EM), early disseminated disease (total of six, including two with multiple EM, one with meningitis, one with isolated facial nerve palsy and one with both facial nerve palsy and multiple EM, one with carditis), and late disseminated disease (five with arthritis). Of these, nine participants had confirmed Lyme disease. Five participants had suspected Lyme disease, including four presenting with a single EM only and negative serology, and one participant with meningitis (reactive Lyme ELISA, one IgM band, and one IgG band, no alternative diagnosis). One participant with unilateral facial palsy had a negative Lyme ELISA (reflex Western blot not performed) and positive herpes simplex virus (HSV) I/II IgM & IgG, providing a potential alternative diagnosis of HSV facial nerve palsy.

All fifteen pediatric cases had mNGS mcfDNA performed, and none met the statistical threshold for B. burgdorferi mcfDNA. Small quantities (1-3 MPM) were detected in four of the samples, including three participants with Lyme arthritis and one diagnosed with HSV facial nerve palsy. B. burgdorferi mcfDNA was detected in 21% (3/14) of suspected or confirmed cases of Lyme disease and in 33% (3/9) of confirmed cases. B. burgdorferi PCR was negative in all nine samples tested.

mNGS detected microbial DNA other than B. burgdorferi in six samples. Of those, one—Adenovirus B—was deemed a potential alternative diagnosis in a patient with fever and carditis. mNGS did not detect mcfDNA from other species of Borrelia or other potential deer tick-borne pathogens, consistent with the lack of clinical suspicion for coinfection.

What is disclosed herein are the results of a pilot study of mNGS of plasma mcfDNA in two cohorts—a validated cohort and a prospective real-world cohort—for the diagnosis of untreated Lyme disease. Investigations of tools that directly detect B. burgdorferi, such as mNGS for mcfDNA, may help to overcome limitations in the diagnostic capabilities for Lyme disease. In the validated cohort NGS for mcfDNA B. burgdorferi mcfDNA was detected in all five confirmed cases and in none of the controls. In the prospective, real-world cohort B. burgdorferi mcfDNA was detected in a subset of participants (33% of participants with confirmed disease), all with Lyme arthritis. Seven out of eight of the B. burgdorferi mcfDNA detections across both cohorts were under the commercial threshold of the mNGS mcfDNA test and were very low in the real world, prospective cohort (1-3 MPM).

The results disclosed herein share similarities and differences with those disclosed by Branda, 2021, Clin Infect Dis, 73: e2355-61 (incorporated herein by reference in its entirety) that investigated the diagnostic utility of mNGS mcfDNA among adults with erythema migrans only. B. burgdorferi DNA was detected at higher rates than disclosed herein, including 48% of all participants and 64% of those with laboratory-confirmed EM. Like the results disclosed herein, detectable levels of B. burgdorferi DNA were low, with a median of 2 MPM, interquartile range of 0-4, and an overall range of <1 to 185 MPM. The rate of samples meeting the predetermined statistical threshold was not reported. Those findings were compared to 3,687 clinical healthy and ill controls previously analyzed, of which B. burgdorferi DNA was detected among two patients (9 and 195 MPM), both with symptoms compatible with Lyme disease.

The apparent absence of any false positive B. burgdorferi DNA detection indicates high test specificity, though the number of control participants at geographic risk of Lyme disease is unclear. In the results disclosed herein, the lack of B. burgdorferi mcfDNA detection in the Case Negatives and the control participants is consistent with the high specificity of B. burgdorferi mcfDNA.

The low B. burgdorferi DNA detection rate in the results herein is likely due to a low pathogen load, the low burden of mcfDNA in localized infections limited to the skin, the ambiguity of defining true positive cases based on clinical symptoms and the limitations of shotgun sequencing. The vast quantity (>99%) of cfDNA in plasma obtained from a human are human cfDNA, not microbial cfDNA. Infections with a low pathogen load, such as Lyme disease, especially acute localized infections limited to the skin are therefore prone to lower mNGS mcfDNA sensitivity. Methods optimized to target, enrich and/or amplify B. burgdorferi DNA prior to sequencing may improve diagnostic performance.

B. burgdorferi blood culture has greater sensitivity in cases of multiple rather than single EM. Hypothetically, mNGS for B. burgdorferi mcfDNA may be more sensitive in patients with acute disseminated EM. In the validated cohort those with disseminated EM had higher levels of B. burgdorferi mcfDNA molecules per microliter than those with a single EM.

The results disclosed herein did not confirm B. burgdorferi as the cause of EM with a punch-biopsy PCR in the prospective pediatric cohort. Rashes may therefore have been due to other causes, including Southern Tick-Associated Rash Illness, though no other pathogens were detected. Given the non-specificity of EM, true positive cases of Lyme disease solely based on clinical signs in the absence of laboratory confirmation are difficult to define.

In summary, B. burgdorferi mcfDNA levels may correlate with Lyme disease in a validated cohort. All three participants with B. burgdorferi detected by mNGS mcfDNA in the prospective cohort had late-stage Lyme arthritis, possibly due to higher sensitivity during that phase of illness, though the quantity of B. burgdorferi mcfDNA detected was quite low. Interestingly, while B. burgdorferi DNA has been confirmed in synovial fluid in the setting of Lyme arthritis, concomitant spirochetemia has not been found. Since PCR requires the presence of intact pathogens (or at least intact microbial genomes), the lack of B. burgdorferi PCR positivity (or B. burgdorferi. blood culture positivity) has been attributed to either the containment of organisms in the joint tissues and/or the lack of viable organisms able to regain access to the blood compartment.

This is the first detection of B. burgdorferi nucleic acid in the plasma of patients with Lyme arthritis and likely represents the fragments of B. burgdorferi mcfDNA undergoing routine metabolism/degradation in the joint space that spill into the plasma compartment. It reflects one of the advantages of using mNGS for mcfDNA as a target analyte in molecular diagnostics—it does not rely on the presence of intact pathogens (or their genomes) and enables a non-invasive means to detect those pathogens even in sequestered locations.

TABLE 1

| | | | | Standard | | |
|---|---|---|---|---|---|---|
| Age | Clinical | B. burgdorferi | Other organisms | two-tier | Lyme disease | Additional |
| (Years) | classification | mcfDNA (MPM) | mcfDNA | Lyme serology | blood PCR | testing notes |
| 4 | Single EM | 0 | H. influenzae | Negative | NP | None |
| 15 | Single EM | 0 | None | Negative | NP | None |
| 4 | Single EM | 0 | None | Negative | NP | None |
| 4 | Single EM | 0 | None | Negative | NP | None |
| 7 | Multiple EM | 0 | None | Positive | NP | None |
| 6 | Multiple EM | 0 | H. influenzae | Positive | Negative | None |
| 10 | Arthritis (knee) | 0 | None | Positive | Negative | Synovial fluid |
| | | | | | | Lyme PCR: positive |
| 7 | Facial nerve palsy | 2 | H. influenzae | Negative | Negative | None; |
| | | | | | | Positive HSV |
| | | | | | | I/II IgM & IgG |
| 10 | Facial nerve palsy | 0 | None | Positive | Negative | None |
| | and multiple EM | | | | | |
| 16 | Meningitis | 0 | H. parainfluenzae | Negative | Negative | CSF Lyme |
| | | | M. cerebrosus | | | PCR not tested |
| | | | N. sicca | | | |
| | | | P. melaninogenica | | | |
| | | | P. nigrescens | | | |
| 6 | Carditis | 0 | Human adenovirus B | Positive | NP | None |
| 13 | Arthritis (knee) | 0 | H. pylori | Positive | Negative | None |
| 3 | Arthritis (knee) | 3 | None | Positive | Negative | Synovial fluid |
| | | | | | | Lyme PCR: positive |
| 11 | Arthritis (knee) | 1 | None | Positive | Negative | Synovial fluid |
| | | | | | | not tested |
| 12 | Arthritis (elbow) | 3 | None | Positive | Negative | Synovial fluid |
| | | | | | | Lyme PCR: negative |

NP—not performed
CSF—cerebral spinal fluid

TABLE 2
Clinical and laboratory findings of study participants with clinical suspicion of Lyme disease.

| | | EM | | | | | | |
|---|---|---|---|---|---|---|---|---|
| Participant | | Size | | B. burgdorferi | Other organisms | Two tier | Two tier | |
| Type | Age | (cm²) | Disseminated | mcfDNA, MPM | mcfDNA (MPM) | ab test V1 | ab test V2 | PCR/ESI-MS |
| Case Positive | 59 | 182 | Yes | 14 | | Positive | Positive | Positive |
| Case Positive | 22 | 169 | Yes | 82 | | Positive | Positive | Positive |
| Case Positive | 66 | 32 | Yes | 173 | | Positive | Positive | Positive |
| Case Positive | 68 | 44 | No | 21 | | Negative | Positive | Positive |
| Case Positive | 32 | 35 | No | 15 | | Negative | Positive | Positive |
| Case Negative | 38 | 36 | No | 0 | S. hominis 136 | Negative | Negative | Negative |
| Case Negative | 63 | 256 | No | 0 | | Negative | Negative | Negative |
| Case Negative | 58 | 32 | No | 0 | | Negative | Negative | Negative |
| Case Negative | 62 | 30 | Yes | 0 | | Negative | Negative | Negative |
| Case Negative | 28 | 36 | No | 0 | | Negative | Negative | Negative |
| Control | 60 | N/A | N/A | 0 | | Negative | Negative | |
| Control | 35 | N/A | N/A | 0 | | Negative | Negative | |
| Control | 26 | N/A | N/A | 0 | | Negative | Negative | |
| Control | 67 | N/A | N/A | 0 | | Negative | Negative | |
| Control | 57 | N/A | N/A | 0 | | Negative | Negative | |

ESI-MS: electrospray ionization mass spectrometry

What is claimed is:

1. A method of treating a Borrelia spp. infection, the method comprising:
(a) providing one or more blood samples from a subject, wherein the blood samples were collected at a time when the subject does not have an erythema migrans (EM) rash, and wherein the one or more blood samples comprise microbial cell-free nucleic acids (mcfNA);
(b) detecting mcfNA from a Borrelia spp. in the one or more blood samples, wherein the mcfNA from the Borrelia spp. detected in the one or more blood samples is indicative of the Borrelia spp. infection in the subject; and
(c) treating the Borrelia spp. infection based, at least in part, on the detecting the mcfNA from the Borrelia spp. in the one or more blood samples.

2. The method of claim 1, wherein the one or more blood samples is one or more plasma samples.

3. The method of claim 1, further comprising quantifying the at least the subset of the mcfNA from the Borrelia spp.

4. The method of claim 1, further comprising attaching nucleic acid adapters to at least a subset of the mcfNA that is from Borrelia spp. to obtain a sequencing library comprising the at least the subset of the mcfNA.

5. The method of claim 4, further comprising generating sequence reads from the sequencing library.

6. The method of claim 5, further comprising aligning the sequence reads to Borrelia spp. genomic sequences in a reference data set to obtain aligned sequence reads.

7. The method of claim 1, further comprising administering to the subject a Borrelia-directed therapy.

8. The method of claim 7, wherein the Borrelia-directed therapy comprises doxycycline, amoxicillin, cefuroxime axetil, ceftriaxone, cefotaxime, or any combination thereof.

9. The method of claim 1, further comprising spiking the one or more blood samples with a known concentration of synthetic DNA.

10. The method of claim 2, further comprising spiking the one or more plasma samples with a known concentration of synthetic DNA.

11. The method of claim 1, wherein the subject has arthritis of a joint.

12. The method of claim 11, wherein the joint comprises a knee joint, an elbow joint, a temporomandibular joint, a hip, or any combination thereof.

13. The method of claim 1, wherein the subject is blood culture negative for Borrelia at the time of the collecting of the one or more blood samples.

14. The method of claim 1, wherein the subject is negative for Borrelia when measured by a polymerase chain reaction (PCR) test of a blood sample or a synovial fluid sample from the subject.

15. The method of claim 1, wherein the subject was bitten by a tick carrying Borrelia bacteria at least 6 months prior to the collecting of the one or more blood samples.

16. The method of claim 1, wherein the subject is serologically positive for Borrelia antibodies.

17. The method of claim 3, wherein a concentration of the at least the subset of the mcfNA from Borrelia spp. is 1-1,000 molecules per microliter (MPM) of plasma.

18. The method of claim 1, wherein the subject has disseminated late-stage Lyme disease.

19. The method of claim 1, wherein a sensitivity of detecting the mcfNA from Borrelia spp. is at least 60%.

20. The method of claim 1, wherein the mcfNA from Borrelia spp. comprises mcfNA derived from B. burgdorferi or B. maynoii bacteria.
